(19) United States
(12) Patent Application Publication
(10) Pub. No.: US 2007/0014830 A1
(43) Pub. Date: Jan. 18, 2007

(54) Drug-releasing sinus stent

(76) Inventors: Edze Jan Tijsma, Maastricht (NL); Claus Bachert, Bellem (BE); James Britton Hissong, Jacksonville, FL (US); Jean-Baptiste HJP Watelet, Eke-Nazareth (BE)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 11/147,929

(22) Filed: Jun. 8, 2005

(51) Int. Cl. A61K 31/65; A61F 2/02

(52) U.S. Cl. 424/426; 514/152

(57) ABSTRACT

The present invention relates to a stent, adapted for deployment in a nasal sinus, comprising a matrix metalloproteinase-inhibiting substance and capable of locally releasing in a controlled manner a therapeutically effective amount of said matrix metalloproteinase-inhibiting substance. The invention further relates to a method for treatment of a diseased or damaged sinus mucosal tissue in a patient, said method comprising introducing into the sinus of said patient a stent comprising a matrix metalloproteinase-inhibiting substance.

### FIELD OF THE INVENTION

The present invention is in the field of wound healing and relates to stents for releasing wound-healing drugs directly to damaged tissues in the paranasal sinus and/or nasal passageways of a patient. The invention further relates to methods of treating sinus disease, and in particular sinusitis.

### BACKGROUND OF THE INVENTION

Sinusitis, the inflammation of the mucosal tissues in the paranasal sinuses, is a common disease that affects humans throughout their lives. In many cases sinusitis is caused by viral infection of the upper respiratory system, but it may also be the result of bacterial or fungal invasion, allergies, medication or structural abnormalities in the paranasal cavities and nasal passageways, genetic defects or intolerance. Sinusitis exists in different forms, the chronic forms being classified as chronic rhinosinusitis (CRS) and nasal polyposis (NP).

The paranasal sinus walls are lined with mucosal tissue. Inflammation of these tissues may lead to blockage of the passageways and the stagnation of mucous may result in bacterial or even fungal infection of the sinus cavities. When symptoms of sinusitis persist and are not responsive to nasal medications, such antibiotic therapy, severe acute sinusitis, CRS and NP may require sinus surgery, which involves opening of sinuses and removal of pathological mucosal tissue.

As an endoscopic technique, Functional Endoscopic Sinus Surgery (FESS) is now the preferred procedure for sinus surgery and for the medical management of CRS and NP. Although the functional results of FESS are satisfactory in the majority of cases, wound healing of the mucosal tissues after FESS is poor in about 20% of patients. This poor healing is associated with abnormal scarring, super-infection, and fibrosis formation, and these complications may in turn lead to recurrence of symptoms and the necessity of revision surgery. Moreover, poor healing may also lead to long-term-complications such as mucoceles, pyoceles, frontal sinusitis, etc.

At present there is a need for a stent that is adapted for deployment in the paranasal cavity, and which is adapted for controlled release of active substances that can improve wound healing after sinus surgery.

### SUMMARY

The present inventors have found that matrix metalloproteinases (MMPs) are involved in the remodeling process of diseased sinus mucosa. They found for instance that protein levels of matrix metalloproteinase-9 (MMP-9; a 92 kDa metalloproteinase also known as gelatinase B) were significantly increased in both CRS and NP diseased states when compared to control values in non-diseased states. They also found that concentrations of MMP-7 (or matrilysin) were significantly increased in NP when compared to controls and CRS, while the protein levels of the tissue inhibitors of metalloproteinase-1 (TIMP-1) were significantly increased in CRS when compared to controls. Furthermore, it was shown by immunohistochemistry that MMPs were expressed in cells lying within zones of tissue destruction, indicating the involvement of MMPs in disease specific remodeling processes. In addition thereto, the present inventors found that high concentrations of MMP-9 in the late postoperative period (1-6 months) were associated with poor healing.

Interestingly, the present inventors further found that preoperative concentrations of MMP-9 in nasal fluid could be used to predict the outcome of the postoperative healing process. This finding was confirmed by the discovery of significantly higher baseline concentrations of nasal fluid MMP-9 in patients that had undergone sinus surgery versus individuals without previous sinus surgery, and demonstrated the impact of previous surgery on the healing outcome. Surgical revision was indicated in patients with persistence or recurrence of symptoms due to abnormal scarring, non-functional mucosa and closure of sinus cavities, with consecutive persistence of disease.

Thus, as a result of their investigations, the present inventors found that MMPs, and in particular MMP-9, may serve as a target for therapeutic intervention in order to achieve the objectives of the present invention and that such therapeutic intervention can be indicated on the basis of preoperative measurements of the nasal fluid concentrations of MMP-9.

Embodiments of the present invention are therefore based on the new insight gained by the inventors that inhibition of matrix metalloproteinase activity in ethmoid and/or frontal sinus tissues can improve the wound healing process of diseased or damaged mucosal tissues, avoid revision surgery, and provide a method of treatment for sinus diseases such as acute sinusitis, chronic rhinosinusitis and nasal polyposis. Embodiments of the present invention now propose for the first time to target metalloproteinases or other factors involved in remodeling or wound healing of the paranasal sinuses.

One embodiment of the present invention provides a stent, adapted for deployment in a paranasal sinus and/or nasal passageway, comprising a matrix metalloproteinase-inhibiting substance and capable of locally releasing in a controlled manner a therapeutically effective amount of said matrix metalloproteinase-inhibiting substance.

In another embodiment, the st [symbol] t is adapted for deployment in the ethmoid sinus and/or frontal sinus.

In yet another embodiment, said matrix metalloproteinase-inhibiting substance inhibits matrix metalloproteinase-9 and/or matrix metalloproteinase-7.

In still another embodiment, said matrix metalloproteinase-inhibiting substance is comprised in a surface coating of said stent. Preferably, said surface coating comprises a polymeric carrier comprising poly (caprolactone), poly (lactic acid), poly (ethylene-vinyl acetate), a copolymer of caprolactone and lactic acid, poly(alpha-hydroxy esters), polyacrylates, ethylene vinyl acetate copolymer or silicone.

In a further embodiment, the stent consists of a sheath forming a hollow body and at least two apertures, said sheath being composed of at least one layer, and wherein at least one layer of said sheath comprises said matrix metalloproteinase-inhibiting substance.

A matrix metalloproteinase-inhibiting substance used in embodiments of the present invention is doxycycline.

In another embodiment, the stent further comprises at least one pharmaceutical agent involved in remodeling processes. The stent is capable of locally releasing in a controlled manner a therapeutically effective amount of said pharmaceutical agent. One or more of the major classes of MMP inhibitor compounds may be used, in particular one or more compounds selected from the group consisting of hydroxamic acids, carboxylic acids, thiols, phosphinic acids, and tetracyclines. Preferred MMP inhibitors include inhibitors selected from the group consisting of N-biphenyl sulfonyl-phenylalanine hydroxamic acid; amines, amino acid derivatives and low molecular weight peptides containing an amide-bound oxal hydroxamic acid moiety; benzodiazepine; acyclic succinic acid-based compounds; oleic acid; cerivastatin; thiol compound MAG-283; tetracycline derivatives, such as tetracycline, doxycycline, and minocycline.

Another embodiment of the present invention provides a method for treatment of a diseased or damaged (para)nasal mucosal tissue in a patient, said method comprising introducing into the paranasal sinus cavity and/or nasal passageway of said patient a stent comprising a matrix metalloproteinase-inhibiting substance and capable of locally releasing in a controlled manner a therapeutically effective amount of said matrix metalloproteinase-inhibiting substance.

In one embodiment of such a method, the sinus mucosal tissue is ethmoid sinus mucosal tissue and/or frontal sinus mucosal tissue

In another embodiment of such a method, the matrix metalloproteinase-inhibiting substance inhibits matrix metalloproteinase-9 and/or matrix metalloproteinase-7. Preferably, said substance is doxycycline or equivalent drugs and/or TIMP-1.

Another embodiment of the present invention relates to a method for treatment of a diseased or damaged sinus mucosal tissue in a patient, said method comprising:

measuring the preoperative concentration of matrix metalloproteinase-9 in nasal fluid;

comparing said concentration with normal baseline levels obtained by measuring the concentration of nasal fluid matrix metalloproteinase-9 in individuals without previous sinus surgery;

optionally performing paranasal sinus surgery on said patient, and

introducing into the paranasal sinus and/or nasal passageway of said patient a stent comprising a matrix metalloproteinase-inhibiting substance and capable of locally releasing in a controlled manner a therapeutically effective amount of said matrix metalloproteinase-inhibiting substance, in case said preoperative concentration of matrix metalloproteinase-9 in the nasal fluid of said patient is above said baseline levels.

In another embodiment of such a method, the sinus mucosal tissue is ethmoid sinus mucosal tissue and/or frontal sinus mucosal tissue and/or nasal passageway tissue.

In another embodiment of such a method, the matrix metalloproteinase-inhibiting substance inhibits matrix metalloproteinase-9 and/or matrix metalloproteinase-7. Preferably, said substance is doxycycline and/or TIMP-1.

### DETAILED DESCRIPTION

A. Definitions

The terms “sinus”, “nasal sinus” and “paranasal sinus”, are used interchangeably herein and are defined as one or more of four pairs of air-filled cavities or cells lined with mucous secreting cells and located within the dense craniofacial bones surrounding the nose, including the frontal, maxillary, ethmoid and sphenoid sinuses. In relation to acute sinusitis, CRS and NP the ethmoidal cleft and frontal sinus are in particular indicated for treatment.

The term “paranasal sinus” indicates an air-filled cavity in the bones of the skull connected to the nasal passageways by small openings (ostia), which allow passage of air to and from the sinus and the drainage of mucous produced by mucosal tissue that lines the sinus walls. The paranasal sinuses are present in four left and right pairs: the frontal sinuses positioned over the eyes in the brow area, the maxillary sinuses inside each cheekbone, the ethmoid sinuses just behind the bridge of the nose and between the eyes, and the sphenoid sinuses behind the ethmoids in the upper region of the nose and behind the eyes.

The terms “paranasal cavity” or “paranasal cavities”, include both the sinus cavities and nasal passageways. The nasal passageways extend from the nasal openings to the choanae, the openings in the roof or soft palate region of the mouth that connect the nasal cavity to the pharynx.

The term “sinus mucosal tissue” includes mucous producing tissue of both the paranasal sinus cavities and nasal passageways.

The term “stent” is used herein in its art-recognized meaning and refers to a spacer or spacing device suitably designed to fit, preferably in self-retaining manner, in a sinus of a patient.

The terms “paranasal stent” or “nasal stent” are used interchangeably herein and refer to a stent designed or adapted for deployment in any of the nasal passageways or paranasal sinuses.

A “patient” for the purposes of the present invention includes both humans and other animals, particularly mammals. Thus the methods are applicable to both human therapy and veterinary applications. In preferred embodiments the patient is a mammal, preferably a primate, and in most preferred embodiments the patient is a human.

The term “metalloproteinase-inhibiting substance” refers to any substance, either chemical or biological, capable of reducing, slowing down or preventing the activity of a metalloproteinase, preferably the activity of a metalloproteinase in vivo, i.e. in the paranasal sinus and/or nasal passageways of a patient. This capability of a substance may for instance be determined ex vivo, e.g. in an experimental setup, wherein the activity of a metalloproteinase is measured in the presence and absence of the potentially inhibiting substance. Measuring metalloproteinase activity is well known in the art, for instance by using colorimetric. Thus, the skilled person is capable of finding known as well as novel metalloproteinase-inhibiting substances.

The term “therapeutically effective amount” as used herein refers to an amount or dose of a therapeutic substance, a matrix metalloproteinase-inhibiting substance, that exerts a detectable therapeutic effect, that improves the healing of wounds to the mucosa of the nasal sinus, in particular after sinus surgery, such as may be performed, by for instance FESS, in relation to complications of acute sinusitis, CRS and/or NP. The term “improve the healing of wounds” is to understood as an improvement in time or quality of the wound healing including the prevention and/or reduction in the occurrence of abnormal scarring, super-infection, and fibrosis formation of such wounds as well as curing diseases and healing damage to affected sinus mucosal tissues. The therapeutic effect can be detected by, for example, imaging or direct observation of mucosal linings of sinuses treated by a method of the invention or contacted with a stent of the present invention by, for instance, endoscopic imaging techniques or by any other suitable method of assessing the progress or severity of sinusitis and sinus mucosal tissue wounds. The precise effective amount for any patient will depend upon the patient's age, body weight, general health, sex, diet, time of administration, drug interaction, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician or experimenter. Methods that permit the clinician to establish initial dosages are known in the art. The dosages determined for administration must be safe and efficacious. The exact dose will depend on the purpose of the treatment, and will be ascertainable by one skilled in the art using known techniques.

Wound healing is a complex, highly integrated and well-coordinated process aimed at closing the wound and, in the case of mucosal wounds, to obtain a new functionally normal mucosa. In general after surgery, various growth factors and enzymes are released from a surrounding tissue into the wound field, including amongst others matrix metalloproteinases. Matrix metalloproteinases are a family of Ca²⁺-activated, zinc-dependent endopeptidases with proteolytic activities towards the different components of the ECM, such as collagen. A range of MMPs are involved in wound healing. Neutrophil-derived matrix metalloproteinase 8 (MMP-8) is the predominant collagenase present in normal healing wounds (Armstrong & Jude, 2002). Remodeling refers to the remodeling process due to trauma or inflammation, indicating that during this process, changes in the tissue structure may occur such as fibrosis, edema, etc. MMPs may degrade Extracellular matrix proteins and may therefore give rise to a repair tissue reaction. This per se is a positive process, however, can lead to a very thick mucosa, if MMPs stay active over a long period of time and prevent cessation of the wound healing process.

The enzymatic properties of MMPs are under strict control of tissue inhibitors of metalloproteinases (TIMPs). TIMPs are highly specific for MMPs and form non-covalent complexes, blocking the access of substrates to the MMP catalytic site. For example, TIMP-1, a natural inhibitor of both MMP-7 and MMP-9, is an inducible soluble protein present in many tissues including nasal mucosa.

B. The Stent

An embodiment of the stent of the present invention is adapted for deployment in a nasal sinus. Thereto, the stent is adapted for introduction into the paranasal sinus of a patient, to be reliably positioned or installed within said sinus and/or to be retained in said sinus. The adaptation may be such that the form (or shape) of the stent is adapted to the anatomy of the sinus for which it is intended and/or the size is adapted to the surface area needed to locally deliver the required dosage of the drug to the intended paranasal sinus. The stent is preferably self-holding through a specific (anatomical) shape or it may be fixed by using known fixation techniques.

A further embodiment of the stent of the present invention comprises a therapeutically effective amount of a matrix metalloproteinase-inhibiting substance, hereinafter also referred to as an MMP-inhibiting substance, details of which are described below.

Another embodiment of the stent of the present invention is further capable of and adapted for locally releasing in a controlled manner a therapeutically effective amount of a matrix metalloproteinase-inhibiting substance. By this it is meant that the stent locally releases medication in an appropriate concentration pattern over time. Controlled release systems typically employ polymeric biomaterials in which the inhibiting substance is entrapped and released into the environment, with release typically occurring through a combination of surface desorption, diffusion and polymer degradation. Controlled release preferably relates to a release of the MMP-inhibiting substance over a predetermined period of time, preferably from 1 week to 12 months, more preferably from 1 to 5 weeks to about 3 to 8 months, even more preferably from about 2-3 weeks to about 2-4 months.

The stent may be prepared from a material comprising a matrix metalloproteinase-inhibiting substance or may consist of a stent body comprising a coating with a matrix metalloproteinase-inhibiting substance. The coating of the stent may comprise or consist of polymers presenting the matrix metalloproteinase-inhibiting substance entrapped in the coating.

The stent may also be prepared from a conventional material such as metal body having a coating loaded with a matrix metalloproteinase-inhibiting substance, said coating being capable of locally releasing in a controlled manner a therapeutically effective amount of a said matrix metalloproteinase-inhibiting substance. Such an embodiment reads on a polymeric release delivery mechanism. A suitable coating material is for instance a crosslinked amphiphilic polymer, such as for instance described in US2004/117006 the disclosures of which is hereby incorporated in its entirety by reference thereto. More details of drug-releasing and optionally expandable stents may for instance be found in U.S. Pat. No. 5,716,981, the disclosure of which is hereby incorporated in their entirety by reference thereto.

As stated, release of matrix metalloproteinase-inhibiting substance from the stents of the invention may occur through drug diffusion, and/or polymer degradation, or a combination of these. For this purpose, the stent may be produced from variety of natural and synthetic materials suitable for release of drugs, which can be categorized as either hydrophobic [e.g., poly(lactide-co-glycolide) (PLG), polyanhydrides] or hydrophilic polymers [e.g., hya [symbol] onic acid (HA), collagen, poly(ethylene glycol) (PEG)]. Synthetic polymers such as PLG and polyanhydrides are very suitable for use in drug delivery applications of the present invention, as they are biocompatible and available in a range of copolymer ratios to control their degradation. Drug release from these polymers typically occurs through a combination of surface desorption, drug diffusion, and polymer degradation.

The stent of an embodiment of the present invention has the form of a hollow tube or hollow body, for instance consisting of a sheath, which forms a hollow body, surrounding an internal cavity. A matrix metalloproteinase-inhibiting substance, which is released in a controlled manner by the stent, may be contained in the sheath or in at least one layer of the sheath, or in a coating covering the outer surface of said sheath. A suitable drug releasing stent of this type is disclosed in US2004/116958, the disclosure of which is hereby incorporated in its entirety by reference thereto.

As suitable stent materials, both organic and inorganic materials, as well as combinations thereof may be used.

Synthetic polymers provide for very suitable organic stent materials. Advantages of such polymers include the ability to tailor mechanical properties and degradation kinetics to suit various applications. Synthetic polymers are also attractive because they can be fabricated into various shapes. Numerous synthetic polymers can be used to prepare synthetic polymer-comprising stents useful in aspects of the invention. They may be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif.

Representative synthetic polymers include alkyl cellulose, cellulose esters, cellulose ethers, hydroxyalkyl celluloses, nitrocelluloses, polyalkylene glycols, polyalkylene oxides, polyalkylene terephthalates, polyalkylenes, polyamides, polyanhydrides, polycarbonates, polyesters, polyglycolides, polymers of acrylic and methacrylic esters, polyacrylamides, polyorthoesters, polyphe [symbol] azenes, polysiloxanes, polyurethanes, polyvinyl [symbol] ohols, polyvinyl esters, polyvinyl ethers, polyvinyl halides, polyvinylpyrrolidone, poly(ether ether ketone)s, silicone-based polymers and blends and copolymers of the above. The stent may comprise both oligomers and polymers of the above.

Specific examples of these broad classes of polymers include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate), poly(vinyl chloride), polystyrene, polyurethane, poly(lactic acid), poly(butyric acid), poly(valeric acid), poly[lactide-co-glycolide], poly(fumaric acid), poly(maleic acid), copolymers of poly (caprolactone) or poly (lactic acid) with polyethylene glycol and blends thereof.

The polymers used in stents may be non-biodegradable. Examples of preferred non-biodegradable polymers include ethylene vinyl acetate (EVA), poly(meth)acrylic acid, polyamides, silicone-based polymers and copolymers and mixtures thereof.

Polymers used in stent may also be biodegradable. The rate of degradation of the biodegradable stent is determined by factors such as configurational structure, copolymer ratio, crystallinity, molecular weight, morphology, stresses, amount of residual monomer, porosity and site of implantation. The skilled person will be able to choose the combination of factors and characteristics such that the rate of degradation is optimized.

Examples of preferred biodegradable polymers include synthetic polymers such as polyesters, polyanhydrides, poly(ortho)esters, polyurethanes, siloxane-based polyurethanes, poly(butyric acid), tyrosine-based polycarbonates, and natural polymers and polymers derived therefrom such as albumin, alginate, casein, chitin, ch [symbol] osan, collagen, dextran, elastin, proteoglycans, gelati [symbol] and other hydrophilic proteins, glutin, zein and other prolamines and hydrophobic proteins, starch and other polysaccharides including cellulose and derivatives thereof (e.g. methyl cellulose, ethyl cellulose, hydroxypropyl cellulose, hydroxy-propyl methyl cellulose, hydroxybutyl methyl cellulose, carboxymethyl cellulose, cellulose acetate, cellulose propionate, cellulose acetate butyrate, cellulose acetate phthalate, cellulose acetate succinate, hydroxypropylmethylcellulose phthalate, cellulose triacetate, cellulose sulphate), poly-l-lysine, polyethylenimine, poly(allyl amine), polyhyaluronic acids, and combinations, copolymers, mixtures and chemical derivatives thereof (substitutions, additions of chemical groups, for example, alkyl, alkylene, hydroxylations, oxidations, and other modifications routinely made by those skilled in the art). In general, these materials degrade either by enzymatic hydrolysis or exposure to water in vivo, by surface or bulk erosion. The foregoing materials may be used alone, as physical mixtures (blends), or as a co-polymer.

Other polymers are polyesters, polyanhydrides, polystyrenes and blends thereof. The polyesters and polyanhydrides are advantageous due to their ease of degradation by hydrolysis of ester linkage, degradation products being resorbed through the metabolic pathways of the body in some cases and because of their potential to tailor the structure to alter degradation rates. The mechanical properties of the biodegradable material are preferably selected such that early degradation and concomitant loss of mechanical strength required for it's functioning as a stent is prevented.

Biodegradable polyesters are for instance poly(glycolic acid) (PGA), poly(lactic acid) (PLA), poly(glycolic-co-lactic acid) (PGLA), poly(dioxanone), poly(caprolactone) (PCL), poly(3-hydroxybutyrate) (PHB), poly(3-hydroxyvalerate) (PHV), poly(lactide-co-caprolactone) (PLCL), poly(valerolactone) (PVL), poly(tartronic acid), poly(β-malonic acid), poly(propylene fumarate) (PPF) (preferably photo cross-linkable), poly(ethylene glycol)/poly(lactic acid) (PELA) block copolymer, poly(L-lactic acid-ε-caprolactone) copolymer, and poly(lactide)-poly(ethylene glycol) copolymers.

Biodegradable polyanhydrides are for instance poly[1,6-bis(carboxyphenoxy)hexane], poly(fumaric-co-sebacic)acid or P(FA:SA), and such polyanhydrides may be used in the form of copolymers with polyimides or poly(anhydrides-co-imides) such as poly-[trimellitylimidoglycine-co-bis(carboxyphenoxy)hexane], poly[pyromellitylimidoalanine-co-1,6-bis(carboph-enoxy)-hexane], poly[sebacic acid-co-1,6-bis(p-carboxyphenoxy)hexane] or P(SA:CPH) and poly[sebacic acid-co-1,3-bis(p-carboxyphenoxy)propane] or P(SA:CPP).

Other suitable stent materials are biocompatible materials that are accepted by the tissue surface. The broad term biocompatible includes also nontoxicity, noncarcinogenity, chemical inertness, and stability of the material in the living body. Exemplary biocompatible materials are titanium, alumina, zirconia, stainless steel, cobalt and alloys thereof and ceramic materials derived therefrom such as ZrO₂ and/or Al₂O₃.

As examples of inorganic stent materials calcium phosphate matrices (CaP) and hydroxyapatite (HA) matrices may be used, wherein HA may optionally be combined with tricalcium phosphate to form such compounds as biphasic calcium phosphate (BCP). CaP, sintered hydroxyapatite and bioactive glasses or ceramics, such as 45S5 Bioglass® (US Biomaterials Corp, USA), and apatite- and wollastonite-containing glass-ceramic (glass-ceramic A-W) may also be used. Very suitable matrix materials are the combined materials such as osteoinductive hydroxyapatite/(HA/TCP) matrices, preferably BCP.

All of the above stent materials may in principle be used in different forms such as in the form of blocks, foams, sponges, sheaths, tubes, granules, cements, coatings, composite components and may for instance consist of combined organic/inorganic materials or ceramics and may be from various origin, natural, biological or synthetic. The various forms may for instance be obtained by extrusion, calendaring, injection moulding, solvent casting, particular leaching methods, compression moulding and rapid prototyping such as 3D Printing, Multi-phase Jet Solidification, and Fused Deposition Modeling (FDM) of the materials. The shape of the stent of the present invention is preferably such that it fits and is retained due to its shape in a particular part of the (para)nasal cavity, preferably a part of the ethmoid sinus and/or frontal sinus, and to leave room for airflow, preferably also for drainage of mucous and/or wound fluid.

C. The MMP-Inhibiting Substance

The MMP inhibiting substance will be chosen based on its inhibitory/antagonizing effect against the MMPs to be targeted. U.S. Pat. No. 5,773,428 to Castelhano et al. and U.S. Pat. No. 5,773,438 to Levy et al. describe certain chemical agents with MMP inhibiting properties, the disclosures of which are hereby incorporated in their entirety by reference thereto.

The MMP inhibiting substance is an MMP-9 and/or MMP-7 inhibiting substance. A suitable example of such a substance is TIMP-1. A preferred MMP inhibiting substance is doxycycline or an MMP inhibiting derivative thereof.

In one embodiment, a therapeutically effective amount, or dose, of an MMP-inhibiting substance is released from the stent and locally administered to a sinus mucosal tissue of a patient. The precise effective amount selected for administration and needed for treating a patient will depend upon various factors as described above. Adjustments for type of sinus disease to be treated, direct contact versus diffusion delivery, and rate of new MMP synthesis, as well as characteristics of the patient as noted above may be necessary.

Stents may be coated with an MMP-inhibiting substance in a variety of manners, including for example: (a) by directly affixing to the stent an MMP-inhibiting substance (e.g., by either spraying the stent with a polymer/drug film, or by dipping the stent into a polymer/drug solution), (b) by coating the stent with a suitable coating polymer such as a hydrogel which will in turn absorb the MMP-inhibiting substance, or (c) by constructing the stent itself with an MMP-inhibiting substance by pre-mixing the MMP-inhibiting substance with the material from which stent is prepared prior to the final preparation of the stent.

D. Therapeutic Treatment

Therapeutic treatment methods of embodiments of the present invention relate to the treatment of paranasal sinus disease, including the treatment of sinusitis, chronic rhinosinusitis (CRS) and nasal polyposis (NP).

A method according to an embodiment the invention for treatment of a patient suffering from a disease of a sinus mucosal tissue comprises the step of introducing into the sinus of said patient a stent comprising a matrix metalloproteinase-inhibiting substance and capable of locally releasing in a controlled manner a therapeutically effective amount of said matrix metalloproteinase-inhibiting substance. The various embodiments of a suitable stent are described above.

Depending on the size and type of the stent and the site of deployment, endoscopic techniques for its introduction may be necessary. Such and other techniques are well within reach of the skilled person.

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. Typically, stents are capable of being compressed, so that they can be inserted through tiny cavities in compressed form and then expanded to a larger diameter when desired, such as when placed at the desired location. A stent of the invention may be self-expanding. Once expanded, the stent physically forces the walls of the passageway apart and holds them open. As such, they are capable of insertion via a small opening, and yet are still able to hold open a large diameter cavity or passageway. The stent may be a frontal sinus stent e.g. the Parell or the Rains frontal sinus stent.

Nasal stents are typically maneuvered into place under direct visual control, taking particular care to place the stent precisely across the narrowing in the cavity being treated.

A method for treatment of a diseased or damaged sinus mucosal tissue in a patient according to an embodiment of the present invention comprises the step of measuring the preoperative concentration of MMP-9 in nasal fluid, comparing said concentration with normal baseline levels obtained by measuring the concentration of nasal fluid MMP-9 in individuals without previous sinus surgery, optionally performing paranasal sinus surgery on said patient, and introducing into the paranasal sinus of said patient a stent comprising a matrix metalloproteinase-inhibiting substance and capable of locally releasing in a controlled manner a therapeutically effective amount of said matrix metalloproteinase-inhibiting substance, in case said preoperative concentration of MMP-9 in the nasal fluid of said patient is above said baseline levels.

Methods for measuring the preoperative concentration of MMP-9 in nasal fluid are known to the skilled person A particularly suitable method consists of collecting nasal fluid by installing a swab or filter paper into the nasal cavity for a certain time (several minutes to several hours) and eluding the fluid therefrom. The fluid retrieved is then used to measure MMP-9 protein by ELISA or equivalent techniques, and the amount of protein will be related to secretion weight.

Normal (healthy) baseline levels of nasal fluid MMP-9 in individuals without previous sinus surgery may be obtained in similar manners as described above. In order to distinguish between a normal baseline level and an elevated concentration of MMP-9, the skilled person will appreciate that comparative values obtained from multiple patients exhibiting poor healing may be used to establish a reference level indicative of elevated concentrations, whereas comparative values obtained from multiple healthy individuals and/or from multiple patients exhibiting good healing may be used to establish a reference level indicative of normal (healthy) baseline levels.

Since the treatment method of an embodiment of the present invention may even prevent the necessity of performing paranasal sinus surgery on said patient, this step is entirely optional. Details on the stent and the introducing thereof into the paranasal sinus of the patient are as described above.

### EXAMPLES

The examples are meant to illustrate one or more embodiments of the invention and are not meant to limit the invention to that which is described below.

### Example 1

Generally, frontal sinus stents are being made by melt processing of a polymer. In such cases, the polymer is processed by extrusion, followed by injection moulding to obtain the material in the shape suitable for placement in the frontal sinus. The commercial PARELL T-STENT® (Medtronic Xomed Surgical Products, Inc., Jacksonville, Fla. USA) is made out of C-FLEX® and is processed by extrusion at 160-200° C., followed by conventional injection moulding at 150-220° C. with injection pressures varying from 300 to 1,000 psi.

The present example describes the manufacture a C-FLEX®-based stent in which an MMP-inhibiting substance is dispersed. Basically, the C-FLEX® is melt is processed with an MMP-inhibiting substance and, optionally, an additive.

In a typical example 400 g of C-FLEX® granules (Consolidated Polymer Technologies, Inc., Clearwater, Fla., USA) were pre-mixed with 50 g of doxycycline hycl [symbol] (Sigma) and 50 g of sodium chloride. Next, this composition was mixed in a twin screw extruder at 160° C. Finally, the extruded material was processed in a screw injection moulding machine at 160° C. to obtain doxycycline-loaded PARELL T-STENT® (˜300 mg weight each). The resulting frontal sinus stent contained 30 mg doxycycline.

### Example 2

The present example describes the application of a coating comprising an MMP-inhibiting substance on a C-FLEX®-based stent. Basically, a medical grade silicone elastomer is formulated with an MMP-inhibiting substance and, optionally, an additive, and the formulation is applied onto a C-FLEX®-based stent.

In a typical example SILASTIC® MDX4-4210 Medical Grade elastomer (Dow Corning corp., Midland, Mich., USA) was used as the coating material: 10 g of MDX4-4210 curing agent was mixed with 100 g of the MDX4-4210 base elastomer. Next, 20 g of doxycycline hyclate and 20 g of sodium chloride was added, and the formulation was thoroughly mixed. The formulation was applied onto PARELL T-STENT® using a brush (˜100 mg on a single T-stent). Finally, the stents were cured in an oven at 110° C. for 60 minutes. The resulting coated frontal sinus stent contained 13 mg doxycycline.

### Example 3

The present example describes the application of a fibre coating comprising an MMP-inhibiting substance on a C-FLEX®-based stent using an electrostatic spinning technique. Basically, a viscous polymer solution is formulated with an MMP-inhibiting substance and, optionally, an additive, and the formulation is applied onto a C-FLEX®-based stent using electrostatic spinning.

In a typical example electrostatic spinning was carried out using solutions of polycaprolactone (Aldrich, M_{w} 80,000) in chloroform. Doxycycline was dissolved in a small amount of methyl alcohol and added to the polymer solution such that the polymer/drug [symbol] eight ratio was 80/20. The electrostatic spinning set-up consisted of a nozzle, a rotating ground electrode onto which a PARELL T-STENT® was mounted, and a high voltage supply. The polymer/drug solution was delivered via a syringe pump to the nozzle, and the solution was deposited as a fibre coating onto the stents (˜25 mg on a single T-stent). The resulting fibre coated frontal sinus stent contained 5 mg doxycycline, and had polymer fibre diameters of ˜1 μm.

What is claimed is:

1. A stent, adapted for deployment in a paranasal sinus and/or nasal passageway, comprising a matrix metalloproteinase-inhibiting substance and capable of locally releasing in a controlled manner a therapeutically effective amount of said matrix metalloproteinase-inhibiting substance.

2. The stent according to claim 1 wherein said nasal sinus is the ethmoid sinus and/or frontal sinus.

3. The stent according to claim 1, wherein said matrix metalloproteinase-inhibiting substance inhibits matrix metalloproteinase-9 and/or matrix metalloproteinase-7.

4. The stent according to claim 1, wherein said matrix metalloproteinase-inhibiting substance is comprised in a surface coating of said stent.

5. The stent according to claim 4, wherein said surface coating comprises a polymeric carrier comprising poly (caprolactone), poly (lactic acid), poly (ethylene-vinyl acetate), a copolymer of caprolactone and lactic acid, poly(alpha-hydroxy esters), polyacrylates, ethylene vinyl acetate copolymer or silicone.

6. The stent according to claim 1, wherein said stent consists of a sheath forming a hollow body and at least two apertures, said sheath being composed of at least one layer, and wherein said at least one layer comprises said matrix metalloproteinase-inhibiting substance.

7. The stent according to claim 1, wherein said matrix metalloproteinase-inhibiting substance is doxycycline and/or TIMP-1.

8. The stent according to claim 1, further comprising at least one pharmaceutical agent involved in remodeling processes.

9. A method for treatment of a diseased or damaged (para)nasal mucosal tissue in a patient, said method comprising introducing into the paranasal sinus and/or nasal passageway of said patient a stent comprising a matrix metalloproteinase-inhibiting substance and capable of locally releasing in a controlled manner a therapeutically effective amount of said matrix metalloproteinase-inhibiting substance.

10. The method according to claim 9, wherein said sinus mucosal tissue is ethmoid sinus mucosal tissue and/or frontal sinus mucosal tissue.

11. The method according to claim 9, wherein said matrix metalloproteinase-inhibiting substance inhibits matrix metalloproteinase-9 and/or matrix metalloproteinase-7.

12. The method according to claim 9, wherein said matrix metalloproteinase-inhibiting substance is doxycycline and/or TIMP-1.

13. A method for treatment of a diseased or damaged sinus mucosal tissue in a patient, said method comprising:
measuring the preoperative concentration of matrix metalloproteinase-9 in nasal fluid;
comparing said concentration with normal baseline levels obtained by measuring the concentration of nasal fluid matrix metalloproteinase-9 in individuals without previous sinus surgery;
optionally performing paranasal sinus surgery on said patient, and
introducing into the paranasal sinus and/or nasal passageway of said patient a stent comprising a matrix metalloproteinase-inhibiting substance and capable of locally releasing in a controlled manner a therapeutically effective amount of said matrix metalloproteinase-inhibiting substance, in case said preoperative concentration of matrix metalloproteinase-9 in the nasal fluid of said patient is above said baseline levels.

14. The method according to claim 13, wherein said sinus mucosal tissue is ethmoid sinus mucosal tissue and/or frontal sinus mucosal tissue and/or nasal passageway tissue.

15. The method according to claim 13, wherein said matrix metalloproteinase-inhibiting substance inhibits matrix metalloproteinase-9 and/or matrix metalloproteinase-7.

16. The method according to claim 13, wherein said substance is doxycycline and/or TIMP-1.
